(19) United States
(12) Patent Application Publication
(10) Pub. No.: US 2018/0021084 A1
(43) Pub. Date: Jan. 25, 2018

(54) RADIO-FREQUENCY ELECTRICAL MEMBRANE BREAKDOWN FOR REDUCING RESTENOSIS

(71) Applicant: RFEMB Holdings, LLC, North Brandford, CT (US)

(72) Inventors: Gary M. Onik, Ft. Lauderdale, FL (US); James A. Miessau, Branford, CT (US); David G. Bostwick, Orlando, FL (US)

(21) Appl. No.: 15/548,935

(22) PCT Filed: Feb. 4, 2016
(86) PCT No.: PCT/US16/16501

§ 371 Date: Aug. 4, 2017

Related U.S. Application Data
(60) Provisional application No. 62/112,059, filed on Feb. 4, 2015.

(51) Int. Cl. A61B 18/14

(52) U.S. Cl. CPC A61B 18/1492; A61B 18/1206

(57) ABSTRACT

An Imaging, guidance, planning and treatment system integrated into a single unit or assembly of components, and a method for using same, that can be safely and effectively deployed to treat re-stenosis from m intra vascular location in various medical settings, including in a hospital or in an outpatient setting. The system utilizes the novel process of Radio-Frequency Electrical Membrane Breakdown (“EMB” or “RFEMB”) to destroy the cellular membranes of unwanted tissue without denaturing the intracellular contents of the cells comprising the tissue, thus preventing or alleviating re-stenosis after an angioplasty-type procedure. The system preferably comprises at least one EMB treatment catheter-type probe 20, at least one temperature sensor 7, and at least one controller unit for at least partially automating the treatment process.

### CROSS-REFERENCE TO RELATED APPLICATIONS

The present invention is a continuation of U.S. Provisional Patent Application Ser. No. 62/112,059, filed Feb. 4, 2015, which is a continuation-in-part of U.S. patent application Ser. No. 14/451,333, filed Aug. 4, 2014, which claims priority to U.S. Provisional Patent Application Nos. 61/912,172, filed Dec. 5, 2013, 61/861,565, filed Aug. 2, 2013, and 61/867,048, filed Aug. 17, 2013, all of which are incorporated herein by reference.

### BACKGROUND OF THE INVENTION

### 1. Field of the Invention

The present invention relates generally to medical devices and treatment methods, and more particularly, to a device and method of utilizing catheter-based radio frequency electrical membrane breakdown (“RFEMB”, or “EMB”) for the prevention of vascular re-stenosis.

### 2. Background of the Invention

Catheters, and more particularly, balloon catheters, have been used to treat stenosis of a vascular or other anatomical tubular structure. In one such procedure, called percutaneous transluminal angioplasty (PTA), a balloon catheter is inserted into a vessel and advanced to the site of the stenosis or lesion where the balloon is inflated against the lesion. Pressure applied to the stenosis by the surface of the inflated balloon compresses the lesion, pushing it radially outward and widening or restoring the luminal diameter of the vessel. Various forms of PTA have been used to treat peripheral arterial stenosis, coronary lesions and other non-vascular tubular structures such as biliary ducts.

Notwithstanding the importance of PTA procedures in restoring normal blood flow to an anatomical region, one problem associated with PTA procedures is the undesired re-growth of the lesion, commonly known as re-stenosis. Re-stenosis, a re-narrowing of the vessel lumen, usually occurs within three to six months after the angioplasty procedure.

Studies have demonstrated a re-stenosis rate after angioplasty in up to 50% of patients treated. Although the use of stents has reduced the re-stenosis rate to approximately 30% of the procedures, re-stenosis remains a significant clinical problem, particularly for those patients whose general health is not conducive to repeat interventional procedures.

The main cause of re-stenosis following angioplasty procedures is due to vessel wall trauma created during the procedure. Evidence has shown that scar tissue forms as endothelial cells that line the inner wall of the blood vessel re-generate in response to the vessel wall injury created during angioplasty. An overgrowth of endothelial cells triggered by the trauma leads to a re-narrowing of the vessel and eventual re-stenosis of the treated area.

Cutting wire balloon catheters, also known in the art, have been used to “score” a stenotic lesion in a more controlled, precise manner. Although it is contemplated that scoring a lesion will lead to less procedural vessel trauma, endothelial cell re-growth and re-stenosis, to date there are no studies that effectively demonstrate this.

Recently, advances in stent technology have included drug-eluting stents which are intended to reduce the occurrence of re-stenosis even further. These types of stents are coated with a drug designed to suppress growth of scar tissue along the inner vessel wall over an extended period of time. The drug is slowly released or eluted, thus reducing the occurrence and extent of re-stenosis when compared with bare stents.

Although shown to be effective in further reducing re-stenosis, there are several known problems with drug-eluting stents including an increased risk in some patient populations of localized blood clots after the drug has been completely eluted, usually after six or more months. Clot formation in the coronary system can lead to heart attack and death.

Therefore, it is desirable to provide a device and method for the prevention of re-stenosis associated with primary angioplasty or placement of a stent that does not require long term administration of drugs to the vessel, as in drug eluting stents.

Irreversible electroporation (IRE) has been proposed as a method for preventing restenosis. See Maor E, Ivorra A, Leor J, Rubinsky B. Irreversible electroporation attenuates neointimal formation after angioplasty, IEEE Trans Biomed Eng. 2008 September; 55(9):2268-74. IRE is a non-thermal ablation modality which ablates the endothelium while leaving the structure of the vessel wall intact.

Irreversible electroporation (IRE) relies on the phenomenon of electroporation. With reference to FIG. 1, electroporation refers to the fact that the plasma membrane of a cell exposed to high voltage pulsed electric fields within certain parameters, becomes temporarily permeable due to destabilization of the lipid bilayer and the formation of pores P. The cell plasma membrane consists of a lipid bilayer with a thickness t of approximately 5 nm. With reference to FIG. 2(A), the membrane acts as a non-conducting, dielectric barrier forming, in essence, a capacitor. Physiological conditions produce a natural electric potential difference due to charge separation across the membrane between the inside and outside of the cell even in the absence of an applied electric field. This resting transmembrane electric potential (V′m) ranges from 40 mv for adipose cells to 85 mv for skeletal muscle cells and 90 mv cardiac muscle cells and can vary by cell size and ion concentration among other things.

With continued reference to FIGS. 2(B)-2(D), exposure of a cell to an externally applied electric field E induces an additional voltage V across the membrane as long as the external field is present. The induced transmembrane voltage is proportional to the strength of the external electric field and the radius of the cell. Formation of transmembrane pores P in the membrane occurs if the cumulative resting and applied transmembrane potential exceeds the threshold voltage which may typically be between 200 mV and 1 V. Poration of the membrane is reversible if the transmembrane potential does not exceed the critical value such that the pore area is small in relation to the total membrane surface. In such reversible electroporation, the cell membrane recovers after the applied field is removed and the cell remains viable. Above a critical transmembrane potential and with longer exposure times, poration becomes irreversible leading to eventual cell death due an influx of extracellular ions resulting in loss of homeostasis and subsequent apoptosis. Pathology after irreversible electroporation of a cell does not show structural or cellular changes until 24 hours after field exposure except in certain very limited tissue types. However, in all cases the mechanism of cellular destruction and death by IRE is apoptotic, which requires considerable time to pass and is not visible pathologically in a time frame to be clinically useful in determining the efficacy of IRE treatment, which is an important clinical drawback to the method.

IRE as an ablation method grew out of the realization that the “failure” to achieve reversible electroporation could be utilized to selectively kill undesired tissue. IRE effectively kills a predictable treatment area without the drawbacks of thermal ablation methods that destroy adjacent vascular and collagen structures. During a typical IRE treatment, one to three pairs of electrodes are placed in or around the tissue. Electrical pulses carefully chosen to induce an electrical field strength above the critical transmembrane potential are delivered in groups of 10, usually for nine cycles. Each 10-pulse cycle takes about one second, and the electrodes pause briefly before starting the next cycle. As described in U.S. Pat. No. 8,048,067 to Rubinsky, et, al and U.S. patent application Ser. No. 13/332,133 by Arena, et al. which are incorporated here by reference, the field strength and pulse characteristics are chosen to provide the necessary field strength for IRE but without inducing thermal effects as with RF thermal ablation.

However, the DC pulses used in currently available IRE methods and devices have some distinct technical and clinical disadvantages when it comes to application of a vascular treatment in an outpatient basis. These include: (1) the need for two different electrodes (positive and negative) built into the treatment device or catheter, which complicates and increases costs in the manufacturing of devices to deliver the treatment and makes the potential use of IRE with stents technically problematic; (2) the need for general anesthesia and neuromuscular blockade due to the severe muscle contractions that are associated with the treatment delivery for IRE; (3) the need for synching the IRE electrical pulses with the cardiac cycle to prevent life threatening ventricular arrhythmias, thus prolonging the treatment; and (4) a tendency for sparking and arcing between the electrodes due to the bipolar nature of the DC pulse, which can cause barotrauma and unwanted vessel damage. Due to these limitations, IRE has thus far not been employed clinically in humans for the stated purpose of vessel restenosis

The propensity of current IRE methods and devices to create severe muscle contraction during treatment is a significant disadvantage because it requires that a patient be placed and supported under general anesthesia with neuromuscular blockade in order for the procedure to be carried out, and this carries with it additional substantial inherent patient risks and costs. Moreover, since even relatively small muscular contractions can disrupt the proper placement of IRE electrodes, the efficacy of each additional pulse train used in a therapy regimen may be compromised without even being noticed during the treatment session.

What is needed is an endothelial ablation method for the prevention of re-stenosis associated with primary angioplasty, or placement of a stent, that does not require long term administration of drugs to the vessel, as in drug eluting stents.

A method that is non-thermal and non-pharmacologic for preventing restenosis would also be advantageous.

In addition, an ablation method that can be accurately targeted at previously identified unwanted stenotic tissue, and that preserves the vascular structure inside of the focal treatment area, would be advantageous.

An ablation modality with the ability to create and monitor a stenotic tissue destruction intravascularly through methods that do not have the inherent limitations of IRE and that does not need neuromuscular blockade, does not cause potentially dangerous sparking would provide a means for preventing restenosis.

It would also be advantageous to provide a system and method for carrying out this treatment on an outpatient basis, under local anesthesia, using a method that does not require general anesthesia or a neuromuscular blockade.

### SUMMARY OF THE INVENTION

It is, therefore, an object of the present invention to provide a method for reducing neointimal hyperplasia and the prevention of vascular re-stenosis using RFEMB via tissue ablation using electrical pulses which causes immediate cell death through the mechanism of complete break down of the cellular membrane of the targeted tissue cells.

It is another object of the present invention to provide such a treatment method that does not require the administration of general anesthesia or a neuromuscular blockade to the patient, so as to provide a system and method for carrying out this treatment on an outpatient basis with treatment probes placed intravascularly under imaging guidance.

It is another object of the present invention to allow the use of non-phamacologic stents to improve blood flow at the same time as the delivery of EMB treatment and as part of the same procedure.

The present invention is an imaging, guidance, planning and treatment system integrated into a single unit or assembly of components, and a method for using same, that can be safely and effectively deployed to prevent vascular restenosis or in an outpatient setting with EMB treatment catheter type probes placed intravascularly under imaging guidance. The invention is comprised of a combination of software, hardware and a process for employing the same through an endoscopic, endoscopic ultrasound, or imaging guided (CT, US, MRI, Flouroscopy) approach. The system utilizes the novel process of Radio-Frequency Electrical Membrane Breakdown (“EMB” or “RFEMB”) to destroy the cellular membranes of unwanted tissue.

In addition, a method of reducing, attenuating or eliminating the intimal formation on a patient that has undergone a surgical procedure in a target area of an artery is disclosed.

The method first involves diagnosing a subject which may be a human subject suffering from coronary artery disease and, specifically, identifying a target area of an artery in the subject that is partially blocked by plaque.

Next, a procedure is performed whereby the blockage in the target area is moved or removed from the artery so as to increase blood flow through the target area of the artery. This procedure can be the placement of an expanding stent, a balloon angioplasty whereby the plaque is forced away from the area of flow, or can involve by-pass surgery whereby the blocked area of the artery is completely removed.

After the procedure is carried out, vascular cells in the area subjected to trauma by the angioplasty or surgery are treated using RFEMB.

The RFEMB treatment may be carried out (1) before, (2) at substantially the same time, or (3) just after the procedure to remove the blockage (e.g. angioplasty) is carried out, but the RFEMB treatment should be carried out before restenosis occurs to obtain the best results.

The RFEMB treatment may be carried out by the use of electrodes which are present on or near the balloon portion of a properly configured balloon catheter used in the angioplasty.

The RFEMB treatment is carried out using a voltage and current within defined ranges over a defined period of time. Further, the RFEMB is carried out in the absence of any drug delivery to the vascular cells in a manner which would effect the growth of the cells.

The method also describes control of the amount of current used and the time for which it is applied to avoid thermal damage.

The result sought per the present invention is to have substantially all of the vascular cells of the targeted area of the artery ablated or killed, but to not raise the temperature of that area sufficiently such as to cause thermal damage and/or denature proteins. By avoiding thermal damage, the structure of the artery and surrounding tissue remains in place. However, due to the disruption of the membrane, the vascular cells are killed and, as such, do not form scar tissue (neointima) in the treatment area, thereby reducing or avoiding restenosis.

The methodology of the invention may involve carrying out the RFEMB treatment at substantially the same time the balloon angioplasty or by-pass surgery is carried out.

It is also possible, according to the present invention, to provide RFEMB treatment prior to or immediately after angioplasty, by-pass surgery or other trauma event, or before or immediately after stent placement.

In addition to the timing, the parameters of RFEMB treatment (voltage/current/pulse duration) are also important. It is undesirable to heat the treatment area, in that too much heat can cause denaturation of the proteins. Denaturation of the proteins results in breakdown of those proteins which thereafter can result in structural breakdown of the vessel, which is also undesirable.

The use of EMB to achieve focal tumor ablation with an enhanced immunologic effect on surrounding cancerous tissue is disclosed in U.S. patent application Ser. No. 14/451,333 and International Patent Application No. PCT/US14/68774, which are both fully incorporated herein by reference.

EMB is the application of an external oscillating electric field to cause vibration and flexing of the cell membrane, which results in a dramatic and immediate mechanical tearing, disintegration and/or rupturing of the cell membrane. Unlike the IRE process, in which nanopores are created in the cell membrane but through which little or no content of the cell is released, EMB completely tears open the cell membrane such that the entire contents of the cell are expelled into the extracellular fluid, and internal components of the cell membrane itself are exposed. EMB achieves this effect by applying specifically configured electric field profiles, comprising significantly higher energy levels (as much as 100 times greater) as compared to the IRE process, to directly and completely disintegrate the cell membrane rather than to electroporate the cell membrane. Such electric field profiles are not possible using currently available IRE equipment and protocols. The inability of current IRE methods and energy protocols to deliver the energy necessary to cause EMB explains why IRE treated specimens have never shown the pathologic characteristics of EMB treated specimens, and is a critical reason why EMB had not until now been recognized as an alternative method of cell destruction.

The system according to the present invention comprises a software and hardware system, and method for using the same, for delivering EMB treatment to the area of the artery which has been affected by stenosis, so that substantially all of the cells in the area are ablated while leaving the structure of the vessel in place and substantially unharmed due to the non-thermal nature of the procedure. The system provides proprietary predictive software tools for designing an EMB treatment protocol to ablate said tissue, and for applying said EMB treatment protocol in an outpatient or hospital setting. The system includes an EMB pulse generator 16, one or more balloon catheter-type EMB treatment probes 20 and one or more temperature probes 22. The system further employs a software-hardware controller unit (SHCU) operatively connected to said generator 16, EMB treatment probes 20 and temperature probe(s) 22, along with one or more optional devices such as endoscopic imaging scanners, ultrasound scanners, and/or other imaging devices or energy sources, and operating software for controlling the operation of each of these hardware devices.

EMB, by virtue of its bipolar wave forms in the described frequency range, does not cause muscle twitching and contraction. Therefore, a procedure using the same may be carried out under local anesthesia without the need for general anesthesia and neuromuscular blockade to attempt to induce paralysis during the procedure. Rather, anesthesia can be applied locally for the control of pain without the need for the deeper and riskier levels of sedation.

In addition, the energy profiles, being bipolar and not DC current, that are used to create EMB also avoid potentially serious patient risks from interference with cardiac sinus rhythm.

In addition, EMB with the applied electrical parameters, does not cause sparking, therefore eliminating the possibility of barotrauma that are associated with IRE.

In addition, since RFEMB can be delivered in a unipolar manner with an indifferent electrode place remotely on the patient, treatment can be delivered after a metal stent is placed, since shorting between the electrodes is not a problem.

### BRIEF DESCRIPTION OF THE DRAWINGS

FIG. 1 is a diagram of a cell membrane pore.

FIG. 2 is a diagram of cell membrane pore formation by a prior art method.

FIG. 3 is a schematic diagram of the software and hardware system according to the present invention.

FIG. 4A is a comparison of a prior art charge reversal with an instant charge reversal according to the present invention.

FIG. 4B is a square wave from instant charge reversal pulse according to the present invention.

FIG. 5 is a diagram of the forces imposed on a cell membrane as a function of electric field pulse width according to the present invention.

FIG. 6 is a diagram of a prior art failure to deliver prescribed pulses due to excess current.

FIG. 7A is a schematic diagram depicting an EMB treatment probe 20 with a built in intravascular US transducer that can be moved into position after an angioplasty is carried out.

FIG. 7B is a schematic diagram depicting the results of a 3D Fused Image of a target tissue.

FIG. 8 is a schematic diagram depicting the target treatment area and Predicted Ablation Zone relative to a therapeutic EMB treatment probe 20 prior to delivering treatment.

FIG. 9 is a schematic diagram of a pulse generation and delivery system for application of the method of the present invention.

FIG. 10 is a diagram of the parameters of a partial pulse train according to the present invention.

FIG. 11 is a schematic diagram depicting the target treatment area and Predicted Ablation Zone relative to a therapeutic EMB treatment probe 20 at the start of treatment delivery.

FIG. 12 is a schematic diagram depicting the positioning of a therapeutic EMB treatment probe 20 comprising an electromagnetic sensor/transmitter 6 according to an embodiment of the present invention proximate the treatment area 2 inside a blood vessel 401.

FIG. 13 is a schematic diagram depicting the positioning of a therapeutic EMB treatment probe 20 comprising a thermocouple 7 according to another embodiment of the present invention proximate the treatment area 2 inside a blood vessel 401.

FIG. 14 is a schematic diagram depicting the positioning of a therapeutic EMB treatment probe 20 comprising a thermocouple 7 according to another embodiment of the present invention proximate the treatment area 2.

FIG. 15 is a schematic diagram depicting the positioning of a therapeutic EMB treatment probe 20 comprising a unipolar electrode 11 according to another embodiment of the present invention proximate the treatment area 2.

FIG. 16 is a schematic diagram depicting the positioning of a therapeutic EMB treatment probe 20 comprising an expandable electrode-bearing balloon 27 according to another embodiment of the present invention inside a blood vessel 401 in the human body.

FIG. 17 is a schematic diagram depicting the positioning of a therapeutic EMB treatment probe 20 comprising an electrode-bearing expandable balloon 27 according to another embodiment of the present invention inside a blood vessel 401 in the human body.

FIG. 18 is a schematic diagram depicting the positioning of a therapeutic EMB treatment probe 20 comprising an insulating sheath 23 bearing electrode 4 according to another embodiment of the present invention inside a blood vessel 401 in the human body.

FIG. 19 is a composite (A & B) schematic diagram depicting the positioning of a therapeutic EMB treatment probe 20 comprising an expandable stent 19 according to another embodiment of the present invention inside a blood vessel 401 in the human body.

FIG. 20 is a schematic diagram depicting the positioning of a stent 19 left by EMB treatment probe 20 inside a blood vessel 401 in the human body.

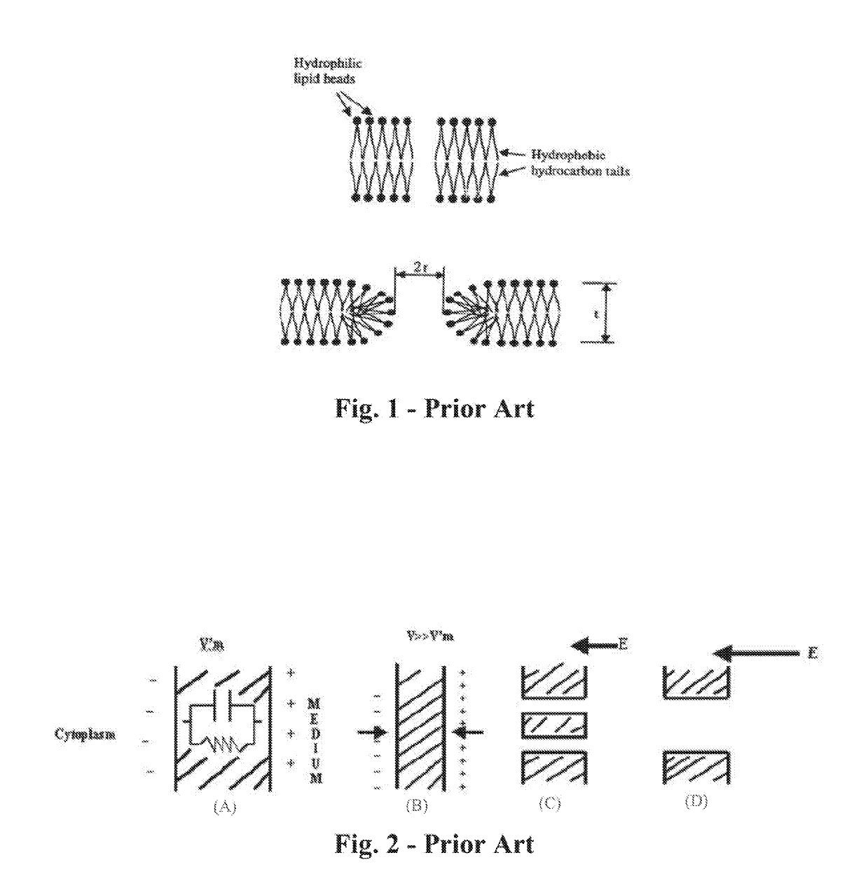

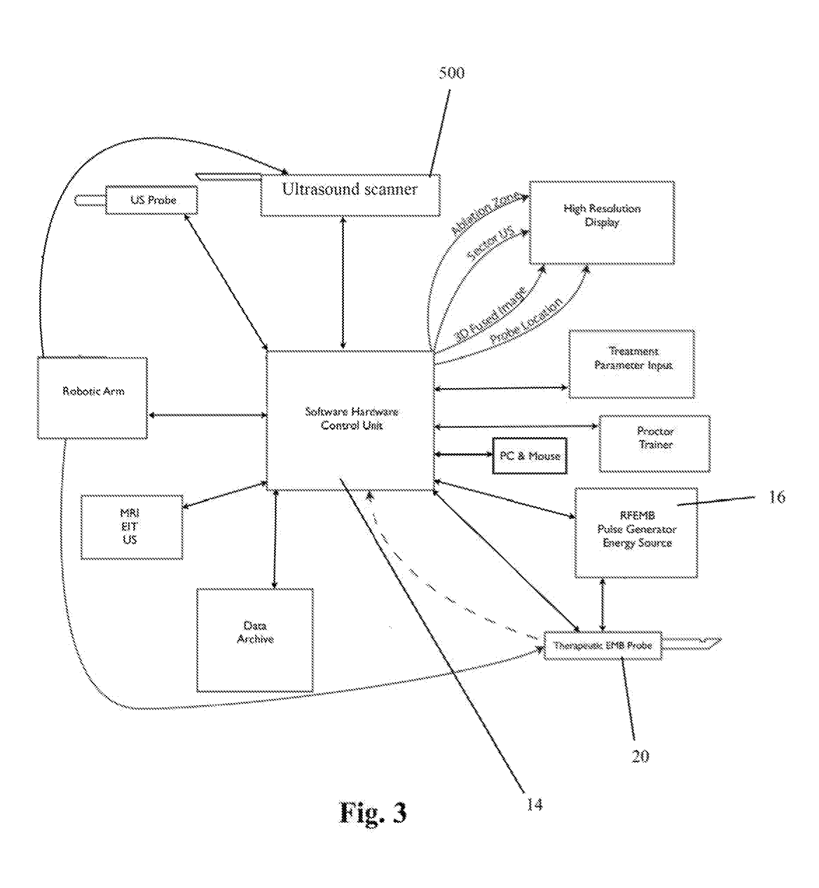

### DETAILED DESCRIPTION

In general, the software-hardware controller unit (SHCU) operating the proprietary catheter-based treatment system software according to the present invention facilitates the treatment of an area of the inner wall of a vessel by directing the placement of EMB treatment probe(s) 20, and by delivering electric pulses designed to cause EMB within the targeted tissue to EMB treatment probe(s) 20, all while the entire process may be monitored in real time via one or more two- or three-dimensional imaging devices. The system is such that the treatment may be performed by a physician under the guidance of the software, or may be performed completely automatically, from the process of imaging the treatment area to the process of placing one or more probes using robotic arms operatively connected to the SHCU to the process of delivering electric pulses and monitoring the results of same. Specific components of the invention will now be described in greater detail.

EMB Pulse Generator 16

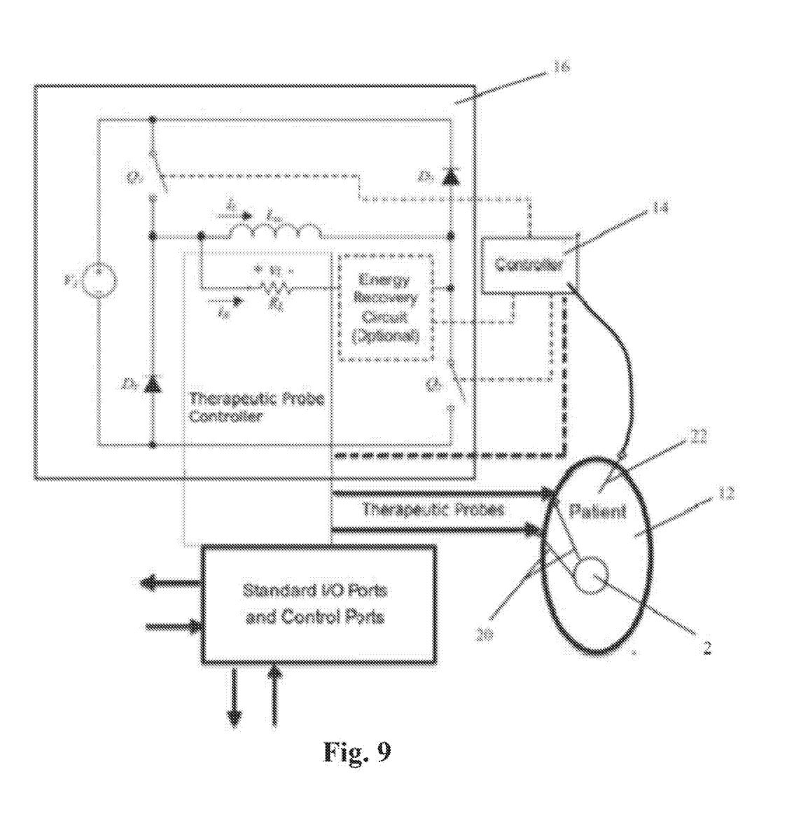

FIG. 9 is a schematic diagram of a system for generation of the electric field necessary to induce EMB of cells 2 within a patient 12. The system includes the EMB pulse generator 16 operatively coupled to Software Hardware Control Unit (SHCU) 14 for controlling generation and delivery to the EMB treatment probes 20 (two are shown) of the electrical pulses necessary to generate an appropriate electric field to achieve EMB. FIG. 9 also depicts optional onboard controller 15 which is preferably the point of interface between EMB pulse generator 16 and SHCU 14. Thus, onboard controller 15 may perform functions such as accepting triggering data from SHCU 14 for relay to pulse generator 16 and providing feedback to SHCU regarding the functioning of the pulse generator 16. The EMB treatment probes 20 (described in greater detail below) are placed in proximity to the vascular lesion treated which are intended to be ablated through the process of EMB and the bipolar pulses are shaped, designed and applied to achieve that result in an optimal fashion. A temperature probe 22 may be provided for temperature measurement on the treatment probe and feedback to the controller of the temperature at, on or near the electrodes. The controller may preferably include an onboard digital processor and a memory and may be a general purpose computer system, programmable logic controller or similar digital logic control device. The controller is preferably configured to control the signal output characteristics of the signal generation including the voltage, frequency, shape, polarity and duration of pulses as well as the total number of pulses delivered in a pulse train and the duration of the inter pulse burst interval.

With continued reference to FIG. 9, the EMB protocol calls for a series of short and intense bi-polar electric pulses delivered from the pulse generator through one or more EMB treatment probes 20 inserted directly into, or placed around the target tissue 2. The bi-polar pulses generate an oscillating electric field between the electrodes that induce a similarly rapid and oscillating buildup of transmembrane potential across the cell membrane. The built up charge applies an oscillating and flexing force to the cellular membrane which upon reaching a critical value causes rupture of the membrane and spillage of the cellular content. Bipolar pulses are more lethal than monopolar pulses because the pulsed electric field causes movement of charged molecules in the cell membrane and reversal in the orientation or polarity of the electric field causes a corresponding change in the direction of movement of the charged molecules and of the forces acting on the cell. The added stresses that are placed on the cell membrane by alternating changes in the movement of charged molecules create additional internal and external changes that cause indentations, crevasses, rifts and irregular sudden tears in the cell membrane causing more extensive, diverse and random damage, and disintegration of the cell membrane.

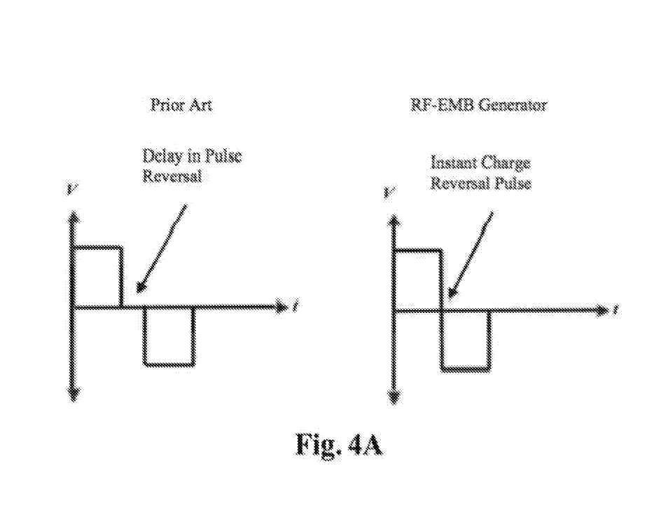

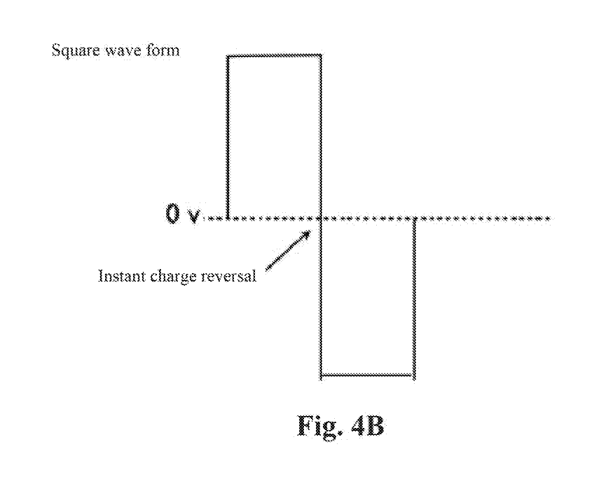

With reference to FIG. 4B, in addition to being bi-polar, the preferred embodiment of electric pulses is one for which the voltage over time traces a square wave form and is characterized by instant charge reversal pulses (ICR). A square voltage wave form is one that maintains a substantially constant voltage of not less than 80% of peak voltage for the duration of the single polarity portion of the trace, except during the polarity transition. An instant charge reversal pulse is a pulse that is specifically designed to ensure that substantially no relaxation time is permitted between the positive and negative polarities of the bi-polar pulse (See FIG. 4A). That is, the polarity transition happens virtually instantaneously.

The destruction of dielectric cell membranes through the process of Electrical Membrane Breakdown is significantly more effective if the applied voltage pulse can transition from a positive to a negative polarity without delay in between. Instant charge reversal prevents rearrangement of induced surface charges resulting in a short state of tension and transient mechanical forces in the cells, the effects of which are amplified by large and abrupt force reversals. Alternating stress on the target cell that causes structural fatigue is thought to reduce the critical electric field strength required for EMB. The added structural fatigue inside and along the cell membrane results in or contributes to physical changes in the structure of the cell. These physical changes and defects appear in response to the force applied with the oscillating EMB protocol and approach dielectric membrane breakdown as the membrane position shifts in response to the oscillation, up to the point of total membrane rupture and catastrophic discharge. This can be analogized to fatigue or weakening of a material caused by progressive and localized structural damage that occurs when a material is subjected to cyclic loading, such as for example a metal paper clip that is subjected to repeat bending. The nominal maximum stress values that cause such damage may be much less than the strength of the material under ordinary conditions. The effectiveness of this waveform compared to other pulse waveforms can save up to ⅕ or ⅙ of the total energy requirement.

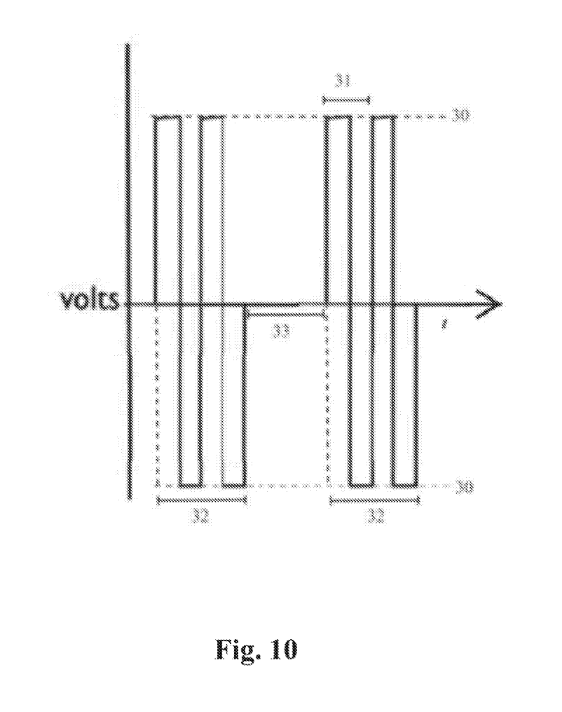

With reference to FIG. 10, another important characteristic of the applied electric field is the field strength (Volts/cm) which is a function of both the voltage 30 applied to the electrodes by the pulse generator 16 and the electrode spacing. Typical electrode spacing for a bi-polar, catheter-type probe might be from 0.75 cm to 1.5 cm. A pulse generator for application of the present invention is capable of delivering up to a 10 kV potential. The actual applied field strength will vary over the course of a treatment to control circuit amperage which is the controlling factor in heat generation, and patient safety (preventing large unanticipated current flows as the tissue impedance falls during a treatment). Where voltage and thus field strength is limited by heating concerns, the duration of the treatment cycle may be extended to compensate for the diminished charge accumulation. Absent thermal considerations, a preferred field strength for EMB is in the range of 1,500 V/cm to 10,000 V/cm.

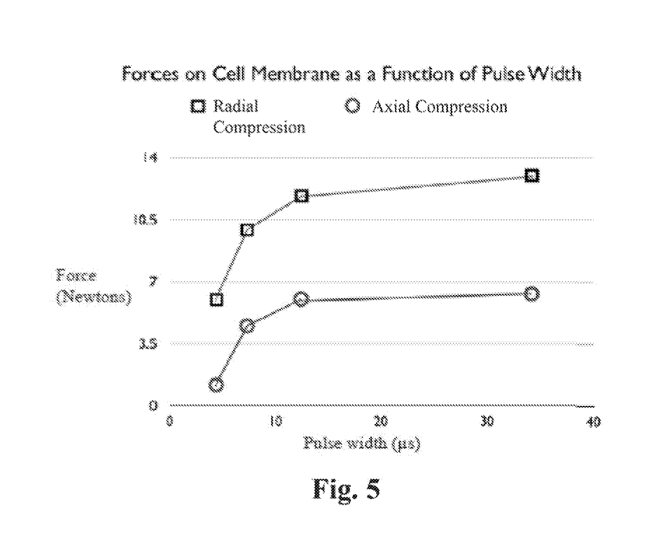

With continued reference to FIG. 10, the frequency 31 of the electric signal supplied to the EMB treatment probes 20, and thus of the field polarity oscillations of the resulting electric field, influences the total energy imparted on the subject tissue and thus the efficacy of the treatment but are less critical than other characteristics. A preferred signal frequency is from 14.2 kHz to less than 500 kHz. The lower frequency bound imparts the maximum energy per cycle below which no further incremental energy deposition is achieved. With reference to FIG. 5, the upper frequency limit is set based on the observation that above 500 kHz, the polarity oscillations are too short to develop enough motive force on the cell membrane to induce the desired cell membrane distortion and movement. More specifically, at 500 kHz the duration of a single full cycle is 2 μs of which half is of positive polarity and half negative. When the duration of a single polarity approaches 1 μs there is insufficient time for charge to accumulate and motive force to develop on the membrane. Consequently, membrane movement is reduced or eliminated and EMB does not occur. In a more preferred embodiment the signal frequency is from 100 kHz to 450 kHz. Here the lower bound is determined by a desire to avoid the need for anesthesia or neuromuscular-blocking drugs to limit or avoid the muscle contraction stimulating effects of electrical signals applied to the body. The upper bound in this more preferred embodiment is suggested by the frequency of radiofrequency thermal ablation equipment already approved by the FDA, which has been deemed safe for therapeutic use in medical patients.

In addition, the energy profiles that are used to create EMB also avoid potentially serious patient risks from interference with cardiac sinus rhythm, as well as localized barotrauma, which can occur with other therapies.

EMB Treatment Probes 20

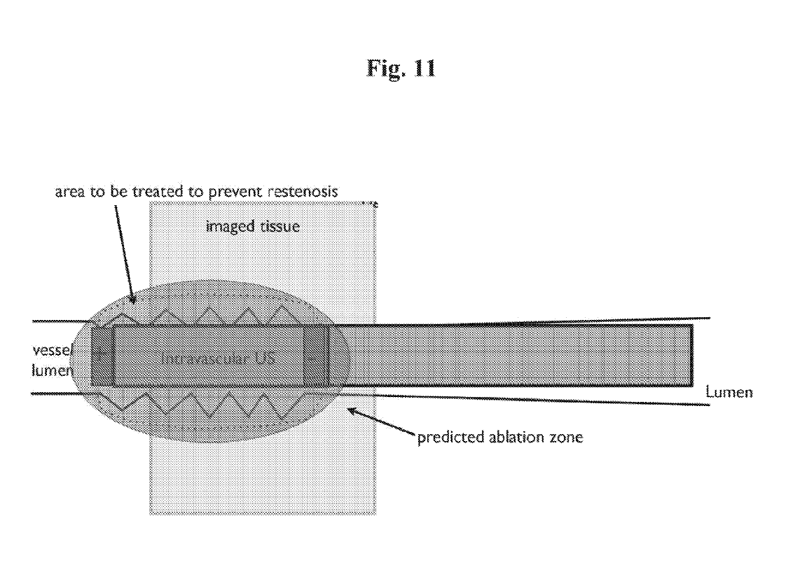

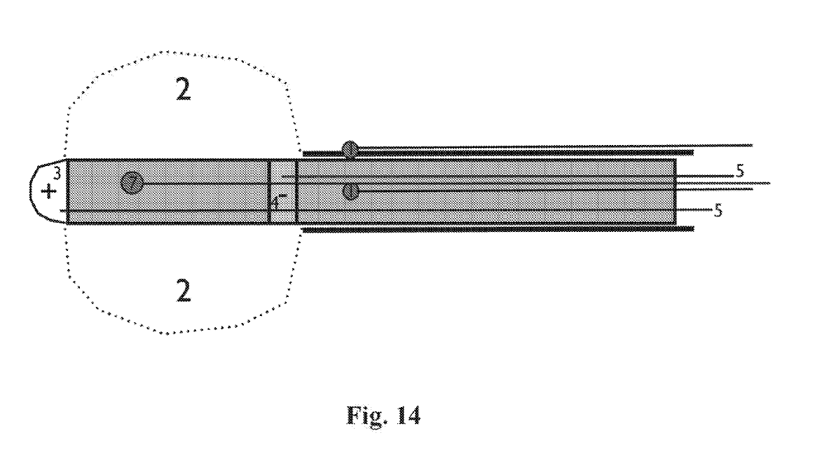

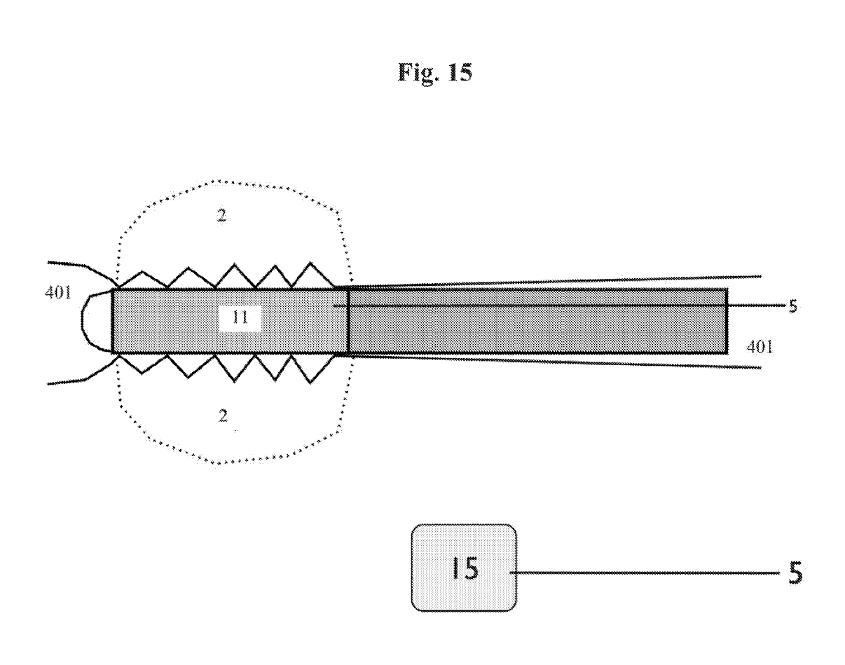

With collective reference to FIGS. 11-20, EMB treatment probes are comprised of at least one therapeutic catheter-type probe 20 capable of delivering therapeutic EMB pulsed radio frequency energy or biphasic pulsed electrical energy under sufficient conditions and with sufficient treatment parameters to completely break down the membranes of the targeted endothelial tissue. Probes 20 are preferably of the catheter type known in the art and having one or more central lumens to, among other things, allow probe 20 to be placed over a guide wire for ease of insertion and/or placement of probe 20 within a blood vessel 401 of the human body according to the Seldinger technique. A catheter for this purpose may be an angiographic balloon type catheter as known in the art, sized between 5 French to 8 French and made of materials generally used for angiographic catheters or any other biocompatible, flexible material.

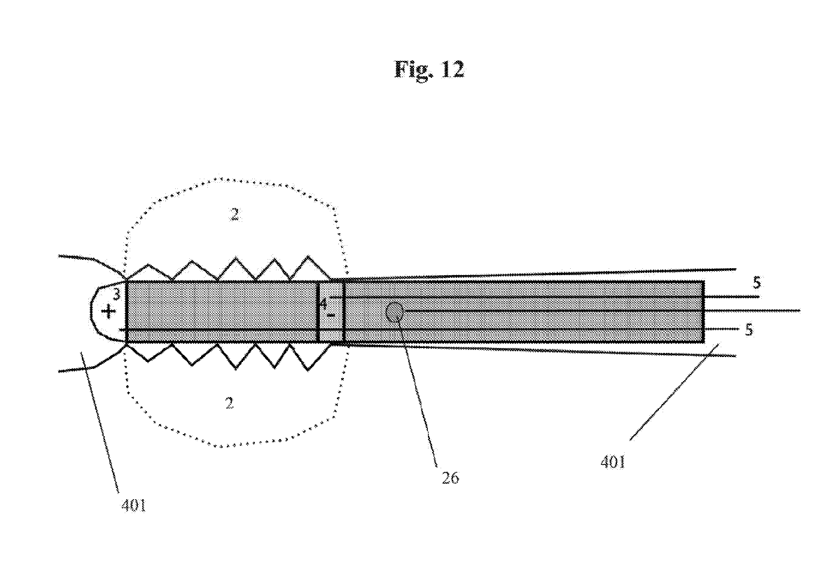

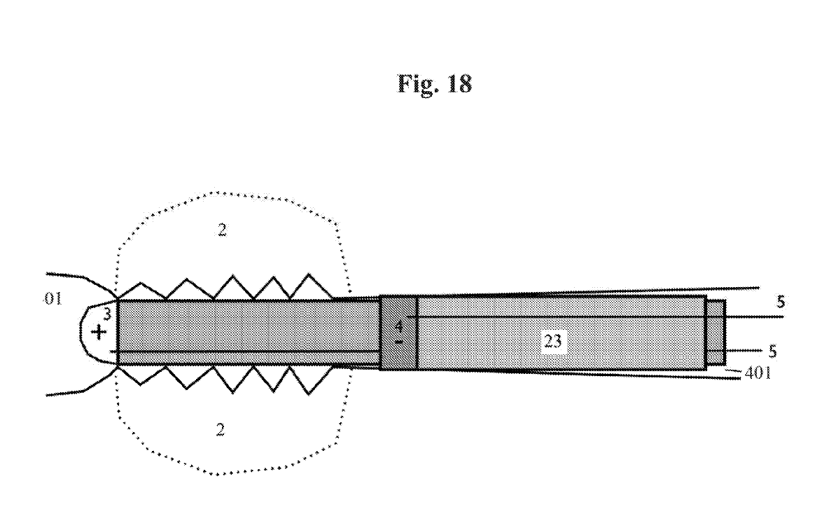

In a preferred embodiment, illustrated in FIG. 12, probe 20 further comprises one positive 3 and one negative 4 electrode disposed on an outer surface of probe 20 and spaced apart by a distance along the longitudinal axis of probe 20 such that current sufficient to deliver the EMB pulses described herein may be generated between the electrodes 3, 4. The spacing between positive 3 and negative 4 electrodes may vary by design preference, wherein a larger distance between electrodes 3, 4 provides a larger treatment area 2. FIG. 12 depicts electrodes 3, 4 on an outer surface of probe 20; alternative, electrodes 3, 4 are integral to the surface of probe 20. In yet another embodiment, as shown in FIG. 18, one of electrodes 3, 4 (negative electrode 4 as shown in FIG. 18) may be placed on the end of an insulated sheath 23 that either partially or fully surrounds probe 20 along a radial axis thereof and is movable along a longitudinal axis of probe 20 relative to the tip thereof (on which positive electrode 3 is located as shown in FIG. 18) to provide even further customizability with respect to the distance between electrodes 3, 4 and thus the size of treatment area 2. Insulating sheath 23 is preferably made of an inert material compatible with bodily tissue, such as Teflon® or Mylar®. One means for enabling the relative movement between probe 20 and insulating sheath 23 is to attach insulating sheath 23 to a fixed member (i.e., a handle) at a distal end of probe 20 opposite the tip of probe 20 by a screw mechanism, the turning of which would advance and retract the insulating sheath 23 along the body of the probe 20. Other means for achieving this functionality of EMB treatment probe 20 are known in the art.

Without limitation, electrodes may be flat (i.e., formed on only a single side of probe 20), cylindrical and surrounding probe 20 around an axis thereof, etc. Electrodes 3, 4 are made of an electrically conductive material. Electrodes 3, 4 may be operatively connected to EMB pulse generator 16 via one or more insulated wires 5 for the delivery of EMB pulses from generator 16 to the treatment area 2. Connection wires 5 may either be intraluminal to the catheter probe 20 or extra-luminal on the surface of catheter probe 20.

Also in a preferred embodiment, as shown in FIG. 12, probe 20 further comprises an electromagnetic (EM) sensor/transmitter 26 that allows visual location of probe 20 within the patient relative to the 3D Fused Image of the treatment area (described in further detail below). EM sensors 26 may be located on both probe 20 and optional insulating sheath 23 to send information to the Software Hardware Controller Unit (SHCU) for determining the positions and/or relative positions of these two elements and thus the size of the treatment area, preferably in real time. EM sensors 26 may be passive EM tracking sensors/field generators, such as the EM tracking sensor manufactured by Traxtal Inc. Alternatively, instead of utilizing EM sensors, EMB treatment probes 20 may be tracked in real time and guided using endoscopy, ultrasound or other imaging means known in the art.

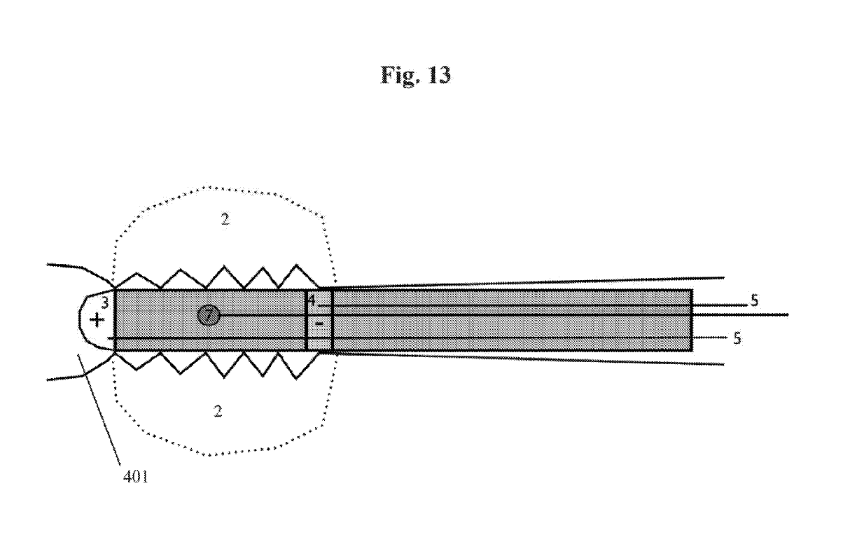

Also in a preferred embodiment, as shown in FIG. 13, probe 20 further comprises a thermocouple 7 on the insulating surface thereof such that the temperature at the wall of the catheter can be monitored and the energy delivery to electrodes 3, 4 modified to maintain a desired temperature at the wall of the probe 20 as described in further detail above. Thermocouple 7 may be, i.e., a Type K-40AWG thermocouple with Polyimide Primary/Nylon Bond Coat insulation and a temperature range of −40 to +180C, manufactured by Measurement Specialties.

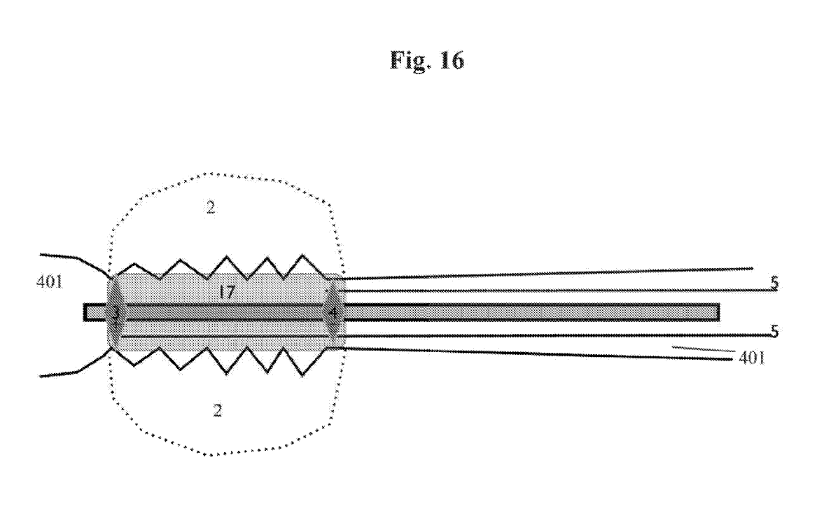

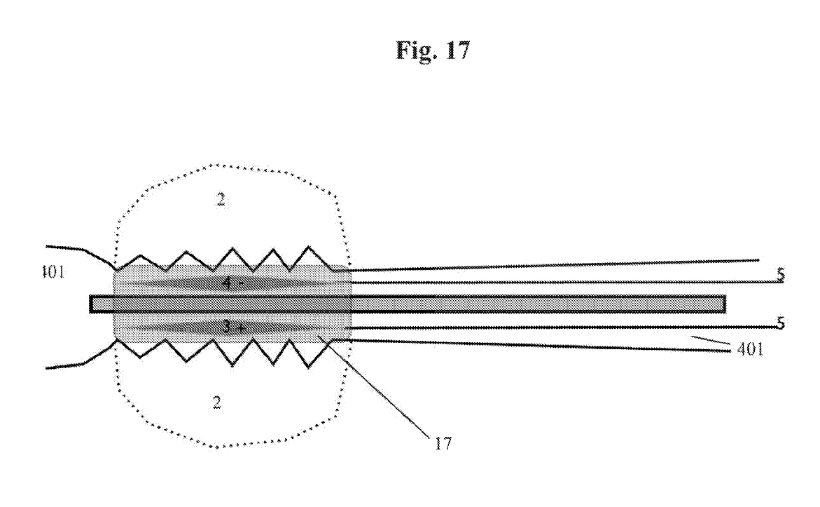

In yet another alternative embodiment of EMB treatment probes 20, unipolar or bipolar electrodes are placed on an expandable balloon 17, the inflation of which may be controlled by the SHCU via a pneumatic motor or air pump, etc. In this embodiment, when the balloon 17 is placed inside a blood vessel 401 in the human body (proximate a designated treatment area) and inflated, the electrodes on the balloon's surface are forced against the wall of the blood vessel 401 to provide a path for current to flow between the positive and negative electrodes (see FIG. 16). The positive and negative electrodes can have different configurations on the balloon 17, i.e., they may be arranged horizontally around the circumference of the balloon 17 as in FIG. 16, or longitudinally along the long axis of the balloon as in FIG. 17. In some embodiments, more than one each of positive and negative electrodes may be arranged on a single balloon.

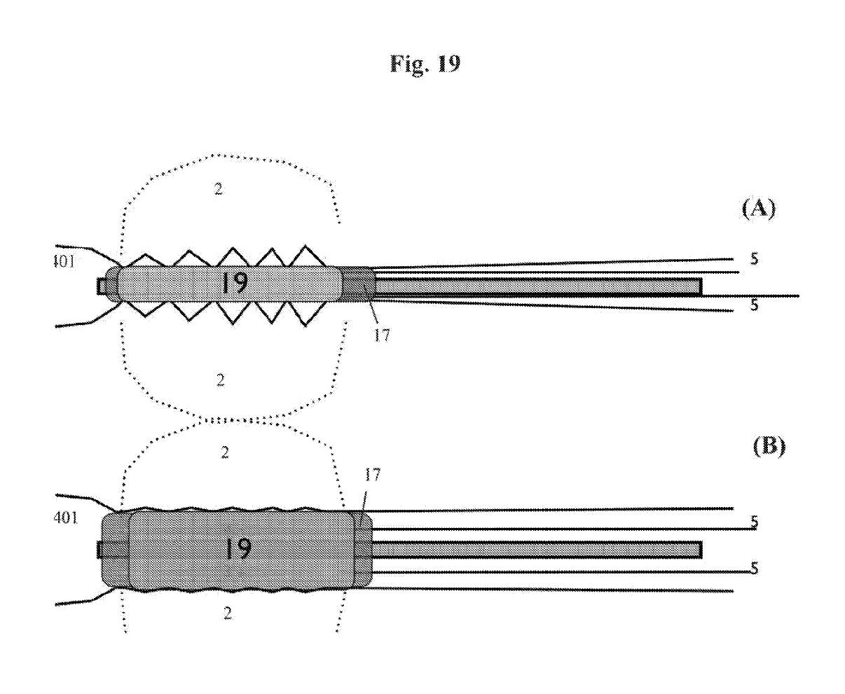

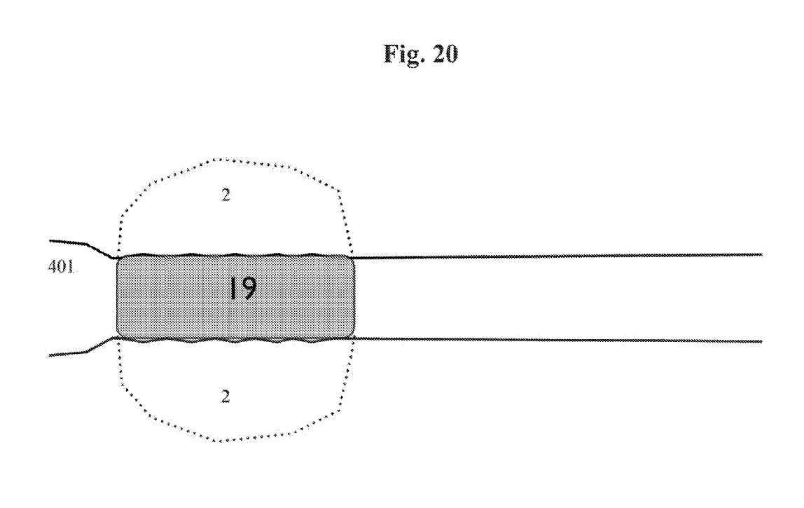

In yet another embodiment, EMB catheter-type probe 20 could deliver a stent 19 to the abnormal region in the blood vessel 401 that is associated with a narrowing causing obstruction. This configuration would allow the delivery of an EMB treatment protocol at the same time as stent 19 is used to expand a stricture in a lumen. Stent 19 may also comprise conducting and non-conducting areas which correspond to the unipolar or bipolar electrodes on EMB probe 20. An example treatment protocol would include placement of EMB probe 20 having balloon 17 with a stent 19 over the balloon 17 in its non expanded state (FIG. 19(A)), expansion of balloon 17 which in turn expands stent 19 (FIG. 19(B)), delivery of the RFEMB treatment, and removal of the EMB treatment probe 20 and balloon 17, leaving stent 19 in place in the patient (see FIG. 20).

In another embodiment, interior lumen 10 may be sized to allow for the injection of biochemical or biophysical nano-materials there through into the EMB lesion to enhance the efficacy of the local ablative effect, or the effect of the EMB treatment, or to allow injection of reparative growth stimulating drugs, chemicals or materials. An interior lumen 10 of the type described herein may also advantageously allow the collection and removal of tissue or intracellular components from the treatment area or nearby vicinity, for any desired testing. This functionality can be used for such purposes before, during or after the application of EMB pulses from the EMB treatment probe 20.

One of ordinary skill in the art will understand that the EMB treatment probe(s) 20 may take various forms provided that they are still capable of delivering EMB pulses from the EMB pulse generator 16 of the type, duration, etc. described above.

EMB, by virtue of its bipolar wave forms in the described frequency range, does not cause muscle twitching and contraction. Therefore a procedure using the same may be carried out under local anesthesia without the need for general anesthesia and neuromuscular blockade to attempt to induce paralysis during the procedure. Rather, anesthesia can be applied locally for the control of pain without the need for the deeper and riskier levels of sedation.

Software Hardware Control Unit (SHCU) 14 and Treatment System Software

With reference to FIG. 3, the Software Hardware Control Unit (SHCU) 14 is operatively connected to one or more (and preferably all) of the therapeutic and/or diagnostic probes, imaging devices and energy sources described herein: namely, in a preferred embodiment, the SHCU 14 is operatively connected to one or more EMB pulse generator(s) 16, EMB treatment probe(s) 20 and temperature sensors 7 via electrical/manual connections for providing power to the connected devices as necessary and via data connections, wired or wireless, for receiving data transmitted by the various sensors attached to each connected device. SHCU 14 is preferably operatively connected to each of the devices described herein such as to enable SHCU 14 to receive all available data regarding the operation and placement of each of these devices.

In an alternative embodiment, SHCU 14 is also connected to one or more of the devices herein via at least one robot arm such that SHCU 14 may itself direct the placement of various aspects of the device relative to a patient, potentially enabling fully automatized and robotic placement and treatment of targeted endothelial tissues via EMB. It is envisioned that the system disclosed herein may be customizable with respect to the level of automation, i.e. the number and scope of components of the herein disclosed method that are performed automatically at the direction of the SHCU 14. At the opposite end of the spectrum from a fully automated system, SHCU 14 may operate software to guide a physician or other operator through a video monitor, audio cues, or some other means, through the steps of the procedure based on the software's determination of the best treatment protocol, such as by directing an operator where to place the EMB treatment probe 20, etc. In each of these variations and embodiments, the system, at the direction of SHCU 14, directs the planning, validation and verification of the Predicted Ablation Zone (to be described in more detail below), to control the application of therapeutic energy to the selected region so as to assure proper treatment, to prevent damage to sensitive structures and/or to provide tracking, storage, transmission and/or retrieval of data describing the treatment applied.

In a preferred embodiment, SHCU is a data processing system comprising at least one application server and at least one workstation comprising a monitor capable of displaying to the operator a still or video image, and at least one input device through which the operator may provide inputs to the system, i.e. via a keyboard/mouse or touch screen, which runs software programmed to control the system in two “modes” of operation, wherein each mode comprises instructions to direct the system to perform one or more novel features of the present invention. The software according to the present invention may preferably be operated from a personal computer connected to SHCU 14 via a direct, hardwire connection or via a communications network, such that remote operation of the system is possible. The two contemplated modes are Planning Mode and Treatment Mode. However, it will be understood to one of ordinary skill in the art that the software and/or operating system may be designed differently while still achieving the same purposes. In all modes, the software can create, manipulate, and display to the user via a video monitor accurate, real-time three-dimensional images of the human body, which images can be zoomed, enlarged, rotated, animated, marked, segmented and referenced by the operator via the system's data input device(s). As described above, in various embodiments of the present invention the software and SHCU 14 can partially or fully control various attached components, probes, or devices to automate various functions of such components, probes, or devices, or facilitate robotic or remote control thereof.

Planning Mode

The SHCU is preferably operatively connected to one or more external imaging sources such as an magnetic resonance imaging (MRI), ultrasound (US), electrical impedance tomography (EIT), or any other imaging device known in the art and capable of creating images of the human body. Using inputs from these external sources, including specifically imaging of the vascular area of the patient's bodily structure to locate suspicious areas that may require treatment, the SHCU first creates one or more “3D Fused Images” of the patient's body in the region of concern. The 3D Fused Images provide a 3D map of the selected treatment area within the patient's body over which locational data obtained from the one or more imaging sources such as an ultrasound scanner according to the present invention may be overlaid to allow the operator to monitor the treatment in real-time against a visual of the actual treatment area.

In a first embodiment, a 3D Fused Image would be created from one or more CT scans and ultrasound image(s) of the same area of the patient's body. A CT image used for this purpose may comprise contrast enhanced CT image created using, i.e., any 64 slice scanner commercially available with standard 3D reconstruction software. In another embodiment, a standard 3D ultrasound known in the art can be used for this purpose. An ultrasound image used for this purpose might be the VH® IVUS (intravascular US) Imaging system using the Eagle Eye® Platinum/Platinum ST RX Digital IVUS Catheter.

The ultrasound image may be formed by, i.e., placing an EM field generator (such as that manufactured by Northern Digital Inc.) on the patient, which allows for real-time tracking of a custom ultrasound probe embedded with a passive EM tracking sensor (such as that manufactured by Traxtal, Inc.).

The 3D fused image is then formed by the software according to the present invention by encoding the ultrasound data using position encoded data correlated to the resultant image by its fixed position to the US transducer by the US scanning device. The software according to the present invention also records of the position of any identified areas of concern for later use in guiding therapy.

This protocol thus generates a baseline, diagnostic 3D Fused Image and displays the diagnostic 3D Fused Image to the operator in real time via the SHCU video monitor. Preferably, the system may request and/or receive additional 3D ultrasound images of the treatment area during treatment and fuse those subsequent images with the baseline 3D Fused Image for display to the operator.

As an alternate means of creating the 3D Fused Image, a two-dimensional sweep of the area is performed in the axial plane to render a three-dimensional ultrasound image that is then registered and fused to a contrast CT or angiogram, or vascular MRA using landmarks common to both the ultrasound image and other reference images. Areas of concern in the vasculature identified on the references images are semi-automatically superimposed on the real-time US image.

The 3D Fused Image as created by any one of the above methods is then stored in the non-transitive memory of the SHCU, which may employ additional software to locate and electronically tag within the 3D Fused Image specific areas of concern that may require treatment, or its vicinity, including sensitive or critical structures and areas. The SHCU then displays the 3D Fused Image to the operator alone or overlaid with locational data from each of the additional devices described herein where available. The 3D Fused Image may be presented in real time in sector view, or the software may be programmed to provide other views based on design preference.

Upon generation of one or more 3D Fused Images of the planned treatment area and, preferably completion of one or more diagnostic imaging scans of the affected area, the SHCU may display to the operator via a video terminal the precise location(s) of one or more areas of concern which require therapy, via annotations or markers on the 3D Fused Image(s): this area requiring therapy is termed the Target Treatment Zone. This information is then used by the system or by a physician to determine optimal placement of the EMB treatment probe(s) 20. Importantly, the 3D Fused Image should also contain indicia to mark the location of treatment targets designated by the physician which will be used to calculate a path to the treatment area. If necessary due to changes in area or tissue size, the geographic location of each marker can be revised and repositioned, and the 3D Fused Image updated in real time by the software, using 3D ultrasound data as described above. The system may employ an algorithm for detecting changes in target tissue size and requesting additional ultrasound scans, and may request ultrasound scans on a regular basis, or the like.

In a preferred embodiment, the software may provide one or more “virtual” EMB treatment catheter type probes 20 which may be overlaid onto the 3D Fused Image showing the areas of concern by the software or by the treatment provider to determine the extent of ablation that would be accomplished with each configuration. Preferably, the software is configured to test several possible probe 20 placements and calculate the probable results of treatment to the affected area via such a probe 20 (the Predicted Ablation Zone) placement using a database of known outcomes from various EMB treatment protocols or by utilizing an algorithm which receives as inputs various treatment parameters such as pulse number, amplitude, pulse width and frequency. By comparing the outcomes of these possible probe locations to the target tissue volume as indicated by the 3D Fused image, the system may determine the optimal probe 20 placement. Alternatively, the system may be configured to receive inputs from a physician to allow him or her to manually arrange and adjust the virtual EMB treatment probes to adequately cover the treatment area and volume based on his or her expertise.

When the physician is satisfied with the Predicted Ablation Zone coverage shown on the Target Treatment Zone based on the placement and configuration of the virtual EMB treatment probes as determined by the system of by the physician himself, the physician “confirms” in the system (i.e. “locks in”) the three-dimensional placement and energy/medication delivery configuration of the virtual EMB treatment probes and the system registers the position of each as an actual software target to be overlaid on the 3D Fused Image and used by the system for guiding the placement of the real probe(s) according to the present invention (which may be done automatically by the system via robotic arms or by the physician by tracking his or her progress on the 3D Fused Image).

If necessary, EMB treatment, as described in further detail below, may be carried out immediately after the planning of therapy is completed for the patient. Alternately, the EMB treatment plan can be created in one session and stored for later use so that EMB therapy may take place days or even weeks later. In the latter case, the steps described with respect to the Planning Mode, above, may be undertaken by the software/physician at any point.

Treatment Mode

The software displays, via the SHCU video monitor, the previously confirmed and “locked in” Target Treatment Zone, Predicted Ablation Zone and 3D Fused Image, with the location and configuration of all previously confirmed virtual probes and their calculated configurations and placements in the vascular location 401, which can be updated as needed at time of treatment to reflect any required changes as described above.

The system preferably displays the Predicted Ablation Zone and the boundaries thereof as an overlay on the 3D Fused Image including the Target Treatment Zone and directs the physician (or robotic arm) as to the intravascular placement of each EMB treatment probe 20. The Predicted Ablation Zone may be updated and displayed in real time as the physician positions each probe 20 to give graphic verification of the boundaries of the Target Treatment Zone, allowing the physician to adjust and readjust the positioning of the Therapeutic EMB Probes, sheaths, electrode exposure and other treatment parameters (which in turn are used to update the Predicted Ablation Zone). When the physician (or, in the case of a fully automated system, the software) is confident of accurate placement of the probes, he or she may provide such an input to the system, which then directs the administration of EMB pulses via the EMB pulse generator 16 and probes 20.

The SHCU controls the pulse amplitude 30, frequency 31, polarity and shape provided by the EMB pulse generator 16, as well as the number of pulses 32 to be applied in the treatment series or pulse train, the duration of each pulse 32, and the inter pulse burst delay 33. Although only two are depicted in FIG. 10 due to space constraints, EMB ablation is preferably performed by application of a series of not less than 100 electric pulses 32 in a pulse train so as to impart the energy necessary on the target tissue 2 without developing thermal issues in any clinically significant way. The width of each individual pulse 32 is preferably from 100 to 1,000 μs with an inter pulse burst interval 33 during which no voltage is applied in order to facilitate heat dissipation and avoid thermal effects. The relationship between the duration of each pulse 32 and the frequency 31 (period) determines the number of instantaneous charge reversals experienced by the cell membrane during each pulse 32. The duration of each inter pulse burst interval 33 is determined by the controller 14 based on thermal considerations. In an alternate embodiment, the system is further provided with a temperature probe 22 inserted proximal to the target tissue 2 to provide a localized temperature reading at the treatment site to the SHCU 14. The temperature probe 22 may be a separate, needle type probe having a thermocouple tip, or may be integrally formed with or deployed from one or more of the Therapeutic EMB Probes 20. The system may further employ an algorithm to determine proper placement of this probe for accurate readings from same. With temperature feedback in real time, the system can modulate treatment parameters to eliminate thermal effects as desired by comparing the observed temperature with various temperature set points stored in memory. This is very important to prevent thermal injury to the inner vessel wall. More specifically, the system can shorten or increase the duration of each pulse 32 to maintain a set temperature at the treatment site to, for example, create a heating (high temp) for the treatment area to prevent bleeding or to limit heating (low temp) to prevent any coagulative necrosis. The duration of the inter pulse burst interval can be modulated in the same manner in order to eliminate the need to stop treatment and maximize the deposition of energy to accomplish EMB. Pulse amplitude 30 and total number of pulses in the pulse train may also be modulated for the same purpose and result.

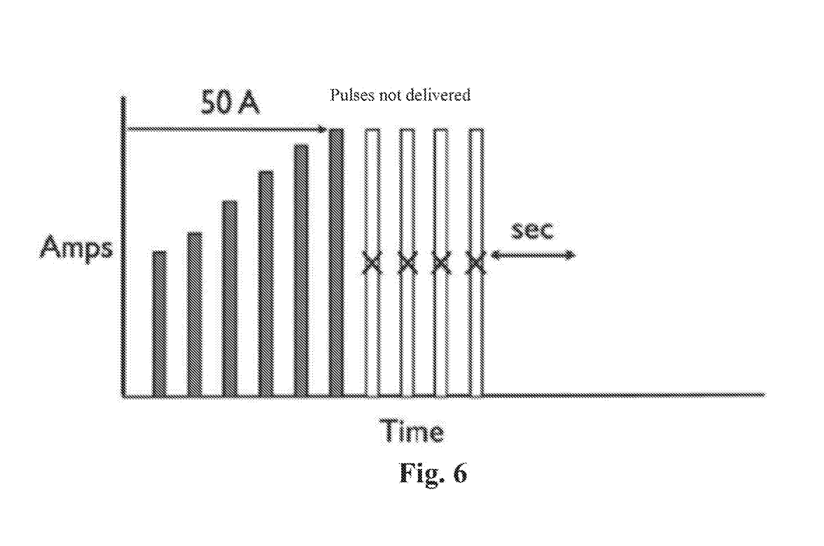

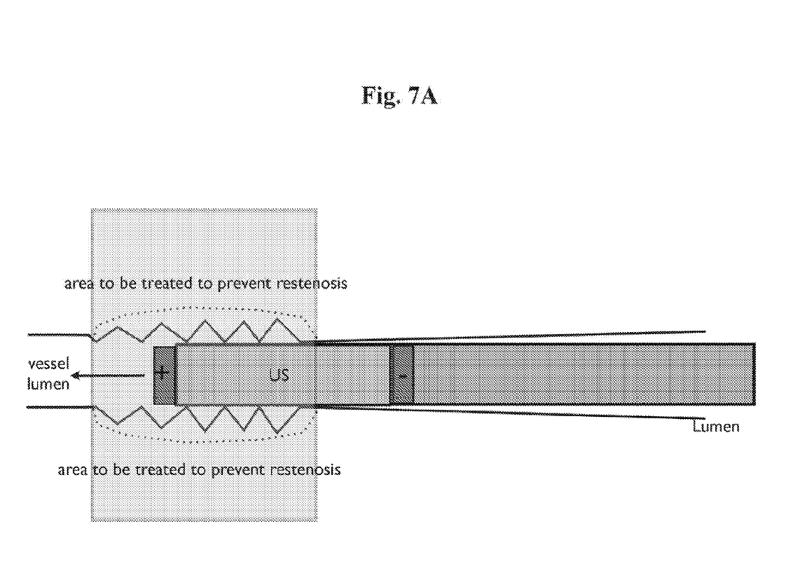

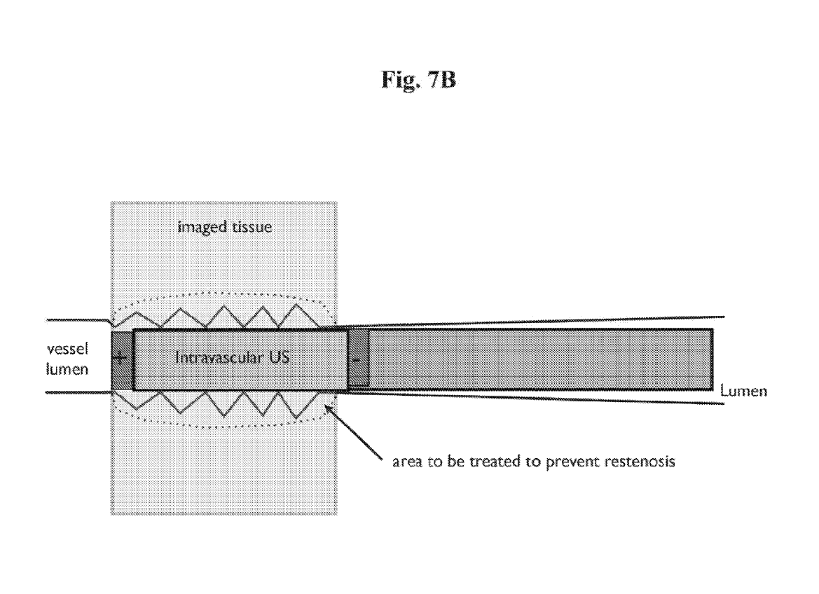

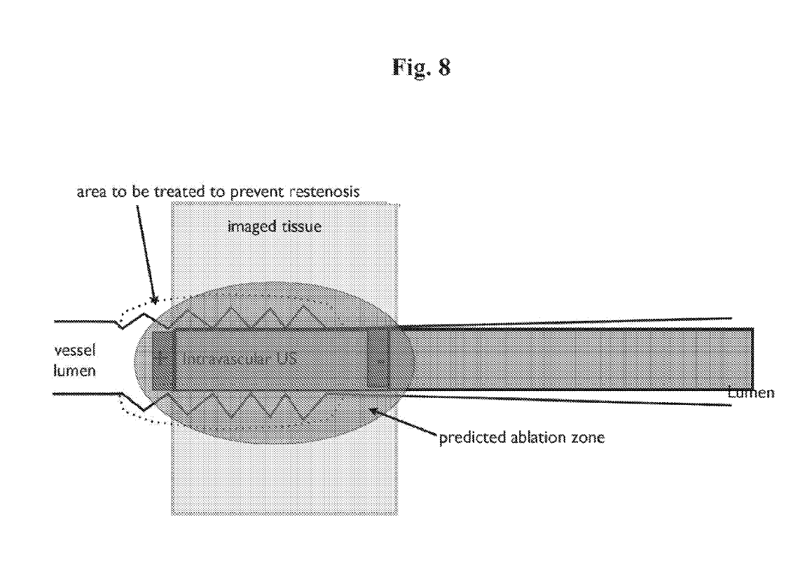

In yet another embodiment, the SHCU may monitor or determine current flow through the tissue during treatment for the purpose of avoiding overheating while yet permitting treatment to continue by reducing the applied voltage. Reduction in tissue impedance during treatment due to charge buildup and membrane rupture can cause increased current flow which engenders additional heating at the treatment site. With reference to FIG. 6, prior treatment methods have suffered from a need to cease treatment when the current exceeds a maximum allowable such that treatment goals are not met. As with direct temperature monitoring, the present invention can avoid the need to stop treatment by reducing the applied voltage and thus current through the tissue to control and prevent undesirable clinically significant thermal effects. Modulation of pulse duration and pulse burst interval duration may also be employed by the controller 14 for this purpose as described.

During treatment, the software captures all of the treatment parameters, all of the tracking data and representational data in the Predicted Ablation Zone, the Target Treatment Zone and in the 3D Fused Image as updated in real time to the moment of therapeutic trigger. Based on the data received by the system during treatment, the treatment protocol may be adjusted or repeated as necessary.

The software may also store, transmit and/or forwarding treatment data to a central database located on premises in the physician's office and/or externally via a communications network so as to facilitate the permanent archiving and retrieval of all procedure related data. This will facilitate the use and review of treatment data, including for diagnostic purposes and pathology related issues, for treatment review purposes and other proper legal purposes including regulatory review.

The software may also transmit treatment data in real time to a remote proctor/trainer who can interact in real time with the treating physician and all of the images displayed on the screen, so as to insure a safe learning experience for an inexperienced treating physician, and so as to archive data useful to the training process and so as to provide system generated guidance for the treating physician. In another embodiment, the remote proctor can control robotically all functions of the system.

Optionally, with one or more EMB treatment probes 20 still in place within the ablated tissue, the physician or system can perform injection of medicines, agents, or other materials into the ablated tissue, using capabilities built into the probe, as described above, or through separate delivery means.

In other embodiments of the present invention, some or all of the treatment protocol may be completed by robotic arms, which may include an ablation probe guide which places the specially designed Therapeutic EMB Probe in the correct intravascular location relative to the target tissue. Robotic arms may also be used to hold the US transducer in place and rotate it to capture images for a 3D US reconstruction.

In addition, the robotic arm can hold the Therapeutic EMB Probe itself and can directly insert the probe into the intravascular location selected for treatment of the target tissue using and reacting robotically to real time positioning data supported by the 3D Fused image and Predicted Ablation Zone data and thereby achieving full placement robotically.

Robotic components capable of being used for these purposes include the iSR′obot™ Mona Lisa robot, manufactured by Biobot Surgical Pte. Ltd. In such embodiments the Software supports industry standard robotic control and programming languages such as RAIL, AML, VAL, AL, RPL, PYRO, Robotic Toolbox for MATLAB and OPRoS as well as other robot manufacturer's proprietary languages.

The SHCU can fully support Interactive Automated Robotic Control through a proprietary process for image sub-segmentation of the targeted tissue and nearby sensitive anatomical structures for planning and performing robotically guided therapeutic interventions.

Sub-segmentation is the process of capturing and storing precise image detail of the location size and placement geometry of the described anatomical object so as to be able to define, track, manipulate and display the object and particularly its three-dimensional boundaries and accurate location in the body relative to the rest of the objects in the field and to the anatomical registration of the patient in the system so as to enable accurate three-dimensional targeting of the object or any part thereof, as well as the three-dimensional location of its boundaries in relation to the locations of all other subsegmented objects and computed software targets and probe pathways. The software sub-segments out various critical substructures, in the treatment region, in a systematic and programmatically supported and required fashion, which is purposefully designed to provide and enable the component capabilities of the software as described herein.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth herein.

### STATEMENT OF INDUSTRIAL APPLICABILITY

Studies have demonstrated a re-stenosis rate after angioplasty in up to 50% of patients treated. Although the use of stents has reduced the re-stenosis rate to approximately 30% of the procedures, re-stenosis remains a significant clinical problem, particularly for those patients whose general health is not conducive to repeat interventional procedures. There would be great industrial applicability in a system or method for the treatment of re-stenosis that reduces this risk, with or without the addition of a stent into the patient, that was minimally invasive and which could be conducted without the need for general anesthesia, which may have dangerous side effects. The instant invention fulfills this need by utilizing Radio-Frequency Electrical Membrane Breakdown to ablate substantially all of the vascular cells of the targeted area of the artery, but to not raise the temperature of that area sufficiently such as to cause thermal damage and/or denature proteins. By avoiding thermal damage, the structure of the artery and surrounding tissue remains in place. However, due to the disruption of the membrane, the vascular cells are killed and, as such, do not form scar tissue (neointima) in the treatment area, thereby reducing or avoiding restenosis.

We claim:

1. A method of treating restenosis in a living subject using radio frequency electrical membrane breakdown, the method comprising:
identifying a location of a vascular blockage in a blood vessel within said living subject;
introducing a catheter-type treatment probe to said location, said catheter-type treatment probe comprising at least one electrode;
removing said vascular blockage from within said blood vessel of said living subject; and applying to an interior surface of said blood vessel at said location, via said at least one electrode, an electric field sufficient to cause electrical membrane breakdown of a cell membrane of a plurality of cells of said soft tissue to cause immediate spillage of all intracellular components into an extracellular space and exposure of an internal constituent part of said cell membrane to said extracellular space.

2. The method of claim 1, wherein said step of removing said vascular blockage occurs prior to said step of applying said electric field.

3. The method of claim 1, wherein said step of removing said vascular blockage occurs after said step of applying said electric field.

4. The method of claim 1, wherein said step of removing said vascular blockage occurs at substantially the same time as said step of applying said electric field.

5. The method of claim 1, wherein said method is performed without administering general anesthesia or a neuromuscular blockade to said living subject.

6. The method of claim 1, wherein said step of removing said vascular blockage comprises placing a stent at said location of said blood vessel.

7. The method of claim 6, wherein said step of placing a stent comprises placing a non-pharmacological stent at said location of said blood vessel.

8. The method of claim 1, wherein said method is performed at least in part by a robotic arm.

9. The method of claim 1, wherein said step of introducing said catheter-type treatment probe to said location comprises:
performing a scan of at least a portion of said living subject;
generating an electronic 3D Fused Image using data obtained from said step of performing a scan;
overlaying at least one virtual catheter-type treatment probe over said 3D Fused Image in one or more configurations;
determining a treatment effect for each of said one or more configurations; determining an optimal placement location of said catheter-type treatment probe based on said treatment effects of said one or more configurations; and
introducing said catheter-type treatment probe into said living subject at said optimal placement location.

10. The method of claim 9, wherein said step of performing a scan comprises performing a CT scan of said living subject.

11. The method of claim 9, wherein said step of generating an electronic 3D Fused Image comprises overlaying the results of a two-dimensional scan of said living subject with the results of a contrast CT scan of said living subject.

12. The method of claim 1, wherein said step of removing said vascular blockage comprises placing a stent in said location of said vascular blockage.

13. The method of claim 12, wherein said stent is metal.

14. The method of claim 1, wherein said step of removing said vascular blockage comprises performing by-pass surgery at said location of said vascular blockage.

15. The method of claim 1, wherein said step of removing said vascular blockage comprises performing a balloon angioplasty at said location of said vascular blockage.

16. A system for treating restenosis in a living subject using radio frequency electrical membrane breakdown, the system comprising:
an electric pulse generator;
at least one therapeutic catheter-type probe comprising at least one electrode operatively connected to said pulse generator, said probe and pulse generator configured to apply to said endothelial cells and intravascular tissue an electric field sufficient to cause electrical membrane breakdown of a cell membrane of a plurality of cells of said soft tissue to cause immediate ablation thereof; and
a controller operatively connected to said electric pulse generator and said therapeutic catheter-type probe.

17. The system of claim 16, said system further comprising at least one thermocouple operatively connected to said controller.

18. The system of claim 16, the system further comprising at least two electrodes, and wherein said at least two electrodes are located at a pre-determined distance from one another on an outer surface of said at least one therapeutic catheter-type probe.

19. The system of claim 16, wherein a first one of said at least one electrodes forms a core of said at least one catheter-type probes, and wherein said at least one catheter-type probe further comprises an insulating sheath comprised of a non-electrically-conductive material surrounding said core on at least one side, wherein a second one of said at least one electrodes is disposed on an outer surface of said insulating sheath.

20. The system of claim 16, wherein said at least one catheter-type probe is a balloon catheter comprising an expandable balloon.

21. The system of claim 20, wherein said at least one catheter-type probe comprises a means for stent delivery.

22. The system of claim 21, wherein said at least one electrode is located on an outer surface of a stent located on said at least one catheter-type probe.

23. The system of claim 20, wherein said at least one electrode is placed on an outer surface of said expandable balloon.

24. The system of claim 16, wherein said at least one catheter-type probe further comprises a central lumen sized to enable injection of materials into said living subject.

25. The system of claim 16, wherein said at least one catheter-type probe further comprises a central lumen sized to enable removal of materials from said living subject.
